(19) United States
(12) Patent Application Publication
(10) Pub. No.: US 2020/0078282 A1
(43) Pub. Date: Mar. 12, 2020

(54) Medicated Cosmetic Formulation

(71) Applicant: Simon Ourian, Beverly Hills, CA (US)

(72) Inventor: Simon Ourian, Beverly Hills, CA (US)

(21) Appl. No.: 16/567,261

(22) Filed: Sep. 11, 2019

Related U.S. Application Data
(60) Provisional application No. 62/729,580, filed on Sep. 11, 2018.

(51) Int. Cl. A61K 8/36; A61K 8/67; A61K 8/368; A61K 8/34; A61K 8/49; A61Q 1/02; A61Q 19/02; A61Q 17/04

(52) U.S. Cl. CPC A61K 8/361; A61K 8/671; A61K 8/368; A61Q 17/04; A61K 8/498; A61Q 1/02; A61Q 19/02; A61K 8/347

(57) ABSTRACT

A medicated cosmetic formulation is provided which comprises one or more vitamin A vitamers, one or more depigmentation agents, and a chemical exfoliant. More particularly, the formulation comprises one or more retinoids as vitamin A vitamers, hydroquinone and/or kojic acid as depigmentation agents, and salicylic acid as a chemical exfoliant. The formulation reduces wrinkles, the appearance of blemishes, and photoaging. In some embodiments, additional ingredients are included, such as skin-colored pigments and ultra violet protection agents. Each of the aforementioned ingredients are provided in ratios that are safe and effective to facilitate collagen production, cell turnover, and depigmentation, and to stimulate blood vessels in the skin.

### CROSS-REFERENCE TO RELATED APPLICATIONS

Pursuant to 35 U.S.C. § 120, this Patent Application relies on the benefit of U.S. Patent App. No. 62/729,580 filed on Sep. 11, 2018. The content of said application is incorporated herein by reference in its entirety.

### GOVERNMENT CONTRACT

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to skin care formulations and, more particularly, to medicated cosmetics which are therapeutically effective cosmetics for reducing wrinkles, blemishes and other signs of aging.

### BACKGROUND

Medicated lotions, serums, cremes, and other treatments are popular for repairing and preventing skin conditions which contribute to the appearance of aging. While there are many such products available on the marketplace, fewer are available as aesthetic makeups, which are popular for their ability to aesthetically conceal such skin conditions as concealer, blush, cosmetic powder, and lipstick.

Additionally, people are frequently admonished to remove makeup and cosmetics from their skin before going to sleep. It is understood that while we sleep, the body, and even the skin, repairs itself and recovers from the stresses of the day. For instance, while a person is sleeping, the skin may heal itself by producing collagen and elastin and forming connective tissue to replace damaged tissues, which each add strength to damaged areas. Blood may deliver additional oxygen and nutrients to light-damaged and other areas of the skin. However, leaving cosmetics and makeup on one's face overnight can clog pores. Clogging pores may interrupt this natural repair process and even aggravate the natural repair process by causing acne, breakouts, and irritation.

In spite of warnings to the contrary, many would like to—and often do, to their detriment—sleep while wearing their cosmetics. For instance, it can be inconvenient and time consuming to fully remove one's makeup application after a long night out or tiring day of work. Some others prefer that their close associates, such as roommates and bed partners, not see them at all without cosmetics enhancing their features or simply evening out their skin tone. Indeed, some overnight skin treatment options available on the marketplace, such as acne treatments, are unsightly when applied to the skin.

Various methods of incorporating antiaging ingredients into cosmetics have been proposed and are incorporated herein by reference. For instance, U.S. Pat. No. 4,159,318 to Mausner et al. teaches oil-free liquid makeup including certain topical medications, U.S. Pat. No. 9,125,919 to Maloney et al., claims a pigmented powder foundation that includes sustained release salicylic acid in its composition along with various salicylate salts to fight acne; and U.S. Pat. No. 6,696,049 to Vatter et al. discloses methods and formulations for improving skin feel of certain makeup which may include some therapeutic ingredients. Still, these do not address all of the benefits of the present invention. Thus, there remains a need for medically therapeutic cosmetics and, additionally, medically therapeutic cosmetics that can be worn to bed.

### SUMMARY

The invention is a variety of topical cosmetic formulations comprising therapeutically effective combinations of retinoids, salicylic acid, hydroquinone, and kojic acid in addition to certain other ingredients such as pigments and protection from ultra violet (“UV”) radiation. The invention may have beneficial properties such as stimulating cell turnover, boosting collagen production, and encouraging moisture retention, which are thought to reduce sign of aging in individuals.

### OBJECTS AND ADVANTAGES

Some objects and advantages of the medicated cosmetic formulations are:

The cosmetic formulation may have medical benefits while aesthetically enhancing a wearer's appearance.

The cosmetic formulation may enable skin cell repair and reduce wrinkles, blemishes and other signs of aging.

The cosmetic formulation may be worn to bed to maintain the aesthetic appearance of the wearer overnight while reducing wrinkles, blemishes and other signs of aging.

Additional objects and advantages will become evidence from the description of the invention.

### DETAILED DESCRIPTION

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested. Additionally, while the disclosure will be below, there is no intent to limit it to the embodiment or embodiments disclosed herein. Rather, the intent is to cover all alternatives, modifications and equivalents included within the spirit and scope of the disclosure.

The cosmetic formulation comprises a combination of ingredients which may reduce wrinkles, the appearance of blemishes, and photoaging. Such ingredients may be provided in ratios that are safe and effective to facilitate collagen production, cell turnover, and depigmentation and to stimulate blood vessels in the skin.

More particularly, the cosmetic formulation comprises a liquid, gel, crème, powder or other material suitable for application to one or more of the face, neck, décolletage, and hands containing, in an amount effective for reducing wrinkles, the appearance of blemishes, and/or photoaging a combination of retinoids, salicylic acid, hydroquinone, and kojic acid. The cosmetic formulation may comprise additional ingredients, such as skin-colored pigments and UV protection, among others, which may superficially contribute to the appearance of an even skin tone and minimize further damage, among other benefits.

In some embodiments, the retinoids may be provided as a vitamin A vitamer to prompt cell turn over and may be selected from compounds comprising synthetic or natural vitamin A such as tretinonin. Other possible retinoids may comprise retinyl esters, retinol, retinaldehyde, and adalpalene. In some embodiments, the retinoid may comprise about 0.025% to about 1.0% of the weight of the composition.

The hydroquinone and kojic acid may comprise some embodiments of the medicated cosmetic formulations as depigmentation agents. One skilled in the art will recognize that either one or both of the hydroquinone and kojic acid may be included in the formulation to practice the invention. In some concentrations, a combination of both hydroquinone and kojic acid may be considered more effective for certain desirable outcomes than either one of the ingredients on their own. In some embodiments, the hydroquinone may comprise about 1% to about 4% of the weight of the composition. In some embodiments, the kojic acid may be about 0.025% to about 2% of the weight of the composition.

Ingredients operative to exfoliate—or in other words cause skin cells to be shed and ultimately increase cell turn over—may also be provided. For instance, a chemical exfoliant known as salicylic acid may be provided. As an oil soluble compound, it is considered effective to penetrate a user's pores, and causing oil and old skin cells clogging such pores to be removed. Salicylic acid may also be effective at reducing skin inflammation and redness. In some embodiments, the salicylic acid may comprise about 0.5% to about 2% of the weight of the composition.

Certain concentrations of possible ingredients comprising the medicated cosmetic formulation have been provided above. However, it is to be understood that any amount of each ingredient effective for any one or more of ultimately reducing wrinkles and blemishes, stimulating cell turnover, boosting collagen production, and encouraging moisture retention in or on the skin is sufficient to practice the invention.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. Accordingly, it is intended that the invention not be limited, except as by the appended claims.

The teachings disclosed herein may be applied to other formulations and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the medicated cosmetic formulation with which that terminology is associated. In general, the terms used in the following claims should not be constructed to limit the medicated cosmetic formulation to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the medicated cosmetic formulation is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the medicated cosmetic formulation.

What is claimed is:

1. A medicated cosmetic formulation comprising:
one or more retinoids;
salicylic acid;
hydroquinone; and
kojic acid.

2. The formulation of claim 1, wherein the one or more retinoids comprises tretinonin.

3. The formulation of claim 1, further comprising:
the one or more retinoids about 0.025 to about 1.0 wt. % of the formulation.

4. The formulation of claim 1, further comprising:
salicylic acid about 0.5 to about 2.0 wt. % of the formulation.

5. The formulation of claim 1, further comprising:
hydroquinone about 1.0 to about 4.0 wt. % of the formulation.

6. The formulation of claim 1, further comprising:
kojic acid about 0.025 to about 2.0 wt. % of the formulation.

7. The formulation of claim 1, further comprising skin-colored pigments and ultra violet protection agents.

8. A cosmetic formulation for reducing wrinkles, blemishes, and photoaging, comprising:
a mixture comprising: one or more retinoids, salicylic acid, and one or more depigmentation agents.

9. The formulation of claim 8, further comprising:
the one or more retinoids about 0.025 to about 1.0 wt. % of the formulation;
salicylic acid about 0.5 to about 2.0 wt. % of the formulation; and
the one or more depigmentation agents 0.025 to about 4.0 wt. % of the formulation.

10. The formulation of claim 8, wherein the one or more depigmentation agents comprise hydroquinone.

11. The formulation of claim 8, wherein the one or more depigmentation agents comprise kojic acid.

12. The formulation of claim 8, further comprising skin-colored pigments and ultra violet protection agents.

13. A medicated cosmetic formulation comprising:
one or more vitamin A vitamers;
one or more depigmentation agents; and
an exfoliant.

14. The formulation of claim 13, wherein the one or more vitamin A vitamers comprise retinoids.

15. The formulation of claim 13, wherein the one or more pigmentation agents comprise hydroquinone and kojic acid.

16. The formulation of claim 13, wherein the exfoliant is a chemical exfoliant.

17. The formulation of claim 16, wherein the chemical exfoliant is salicylic acid.

18. The formulation of claim 13, further comprising skin-colored pigments and ultra violet protection agents.
